(19) United States
(12) Patent Application Publication
(10) Pub. No.: US 2017/0238824 A1
(43) Pub. Date: Aug. 24, 2017

(54) METHOD FOR OSCILLATORY NON-INVASIVE BLOOD PRESSURE (NIBP) MEASUREMENT AND CONTROL UNIT FOR AN NIBP APPARATUS

(71) Applicant: KONINKLIJKE PHILIPS N.V., EINDHOVEN (NL)

(72) Inventors: Pierre Hermanus WOERLEE, VALKENSWAARD (NL); Paul AELEN, EINDHOVEN (NL)

(73) Assignee: KONINKLIJKE PHILIPS N.V., Eindhoven (NL)

(21) Appl. No.: 15/503,023

(22) PCT Filed: Aug. 19, 2015
(86) PCT No.: PCT/EP2015/068978

§ 371 Date: Feb. 10, 2017

(30) Foreign Application Priority Data
Aug. 28, 2014 (EP) 14182676.8

(51) Int. Cl. A61B 5/022

(52) U.S. Cl. CPC A61B 5/02225; A61B 2562/0247

(57) ABSTRACT

There is provided a method for use in cuff-based oscillatory non-invasive blood pressure (NIBP) measurement. The method comprises: progressively altering the volume of air in a cuff of a NIBP measurement apparatus during a measurement period; obtaining a plurality of measurements of the flow rate of the air into/out of the cuff during the measurement period; obtaining a plurality of measurements of the air pressure in the cuff during the measurement period; and determining a relationship between quasi-static cuff compliance and cuff pressure by calculating the quasi-static cuff compliance at a plurality of instances during the measurement period, based on the flow rate measurements and the air pressure measurements obtained during the measurement period.

### TECHNICAL FIELD OF THE INVENTION

The invention relates to a method for use in cuff-based oscillatory non-invasive blood pressure (NIBP) measurements and a control unit for an NIBP apparatus, and in particular relates to a method for acquiring oscillatory NIBP measurements with a minimal error and a control unit which enables an NIBP apparatus to implement the method.

### BACKGROUND TO THE INVENTION

Arterial blood pressure (BP) is one of the most important vital signs and is widely used in clinical practice. Non-invasive arterial blood pressure (NIBP) is usually measured by slowly varying the pressure in a cuff that is wrapped around the upper arm of a subject. The NIBP is determined either by measuring sound distal from the cuff (the auscultatory method, based on Korotkoff sounds) or by measuring pressure pulsations in the cuff caused by volume pulsations of the arm and brachial artery and extracting features from the envelope of these pressure pulses (the oscillometric method). The oscillometric method is easily automated and is widely used. However, the auscultatory method is the “gold standard” for cuff based NIBP measurements. The deviation between the results yielded by the auscultatory method and the results yielded by any other BP measurement method should comply with NIBP standards (set by the British Hypertension Society in the UK and by the Association for the Advancement of Medical Instrumentation (AAMI) in the US).

The principle behind the oscillometric method is illustrated by FIG. 1, which shows a graph of cuff pressure 10, and a processed high pass filtered trace 12 of this cuff pressure, versus time. The left-hand y-axis shows pulse amplitude, the right-hand y-axis shows cuff pressure, and the x-axis shows time. To perform NIBP measurement using the oscillometric method, first the cuff pressure 10 is ramped up until it is sufficiently larger than systolic blood pressure. After ramp up, the cuff is deflated (in FIG. 1 the deflation is done gradually, but step wise deflation is also possible). During the deflation, small oscillations in cuff pressure occur, caused by volume changes in the bladder of the cuff, which are in turn caused by volume changes in the brachial artery. The measured cuff pressure 10 is high pass filtered, and the resulting trace 12 shows the cuff pressure oscillations due to volume changes in the brachial artery. An envelope 14 of the oscillation amplitudes is determined. The maximum Aₘₐₓ of this pulse envelope 14 is taken as a reference point for determining the systolic 16 and diastolic pressure 15. The systolic pressure 16 is determined as the cuff pressure where the pressure oscillation is approximately 0.8 times the maximum amplitude Aₘₐₓ at a pressure higher than the pressure at the reference point. The diastolic pressure 15 is determined as the cuff pressure where the pressure oscillation is approximately 0.55 times the maximum amplitude Aₘₐₓ at a pressure lower than the pressure at the reference point. These ratios are based on empirical values (see, e.g., L A Geddes et. al., Annals of Biomedical Engineering 10 pp 271-280, 1982). The exact algorithms that are employed by manufacturers of blood pressure devices to determine systolic and diastolic pressures are usually trade secrets.

The typical apparatus 20 used for acquiring oscillometric NIBP measurements is illustrated in FIG. 2. A pump 21, first and second pressure sensors 22, 23, and first and second valves 24, 25 are connected to a cuff 26 by tubing 27. During execution of the oscillometric method the pump 21 blows air into the cuff 26, thereby inflating it. The first and second pressure sensors 22, 23 measure the pressure in the system (and therefore the pressure in the cuff 26). When a pressure larger than systolic pressure is reached, the pump 21 is disabled, the first valve 24 is opened and slow (or step wise) deflation occurs, during which the cuff pressure is continuously measured and the measurements stored. The pump and valves are controlled by a control unit (not shown), which also receives the cuff pressure measurements and calculates the pulse envelope and the systolic and diastolic pressure using these measurements. Multiple sensors and valves are used for safety reasons.

Oscillatory blood pressure measurements can have large errors (10 s of mmHg, corresponding to 10 s of percent), both for subjects with low blood pressure and for subjects with high blood pressure (see, e.g., Wax D B et. al., Anaesthesiology 115 pp 973-978, 2011). The errors are due to systematic flaws associated with using a cuff. Error sources include, for example:

1) Pressure drop over the (visco-elastic) wall of the cuff;
2) Pressure transmission over the soft tissue;
3) Size of the cuff;
4) Variation in the mechanical properties of the arm between different subjects;
5) Variation in arm size between different subjects;
6) Variation in cuff placement;
7) Variation in characteristics of the cuff (i.e. pressure dependent compliance);
8) Tube flow resistance and pressure drop over the tube.

A key source of error is the non-constant value of pressure dependent quasi-static (QS) cuff compliance. Cuff compliance C_{C} is the value that relates the pressure change in the cuff due to a volume change of the cuff when the number of air particles in the cuff is constant and elasticity of the cuff wall is negligible, it is represented by the function

$${C}_{C} ( {P}_{C} ) = \frac{{dV}_{C}}{{dP}_{C}} \qquad ( 1 )$$

where V_{C} is the volume of the cuff and P_{C} is the pressure in the cuff. In first order it can be calculated using Boyle's Law. The compliance function varies depending on the pressure in the cuff; on how exactly the cuff is wrapped around a subject's arm and also with the size and mechanical properties of the arm. The pressure in the cuff has a significant influence on the cuff compliance.

FIG. 4 shows a plot of a measured cuff compliance versus cuff pressure for a particular adult cuff. At high cuff pressure (>100 mmHg) cuff compliance is nearly constant but at low pressure the compliance is strongly dependent on pressure. This gives rise to errors in the pressure oscillation-amplitude measurement, as for a given volume change in the cuff, the pressure change depends on cuff compliance. In order to estimate volume oscillations in a proper way, the cuff compliance should be constant for all cuff pressures, which is clearly not the case, especially at low cuff pressures. From FIG. 4 it is clear that the transfer function (equation 1) is not constant, which means that distortion of the pulse pressure envelope can occur, especially in low pressure regions (<60 mmHg), e.g. in subjects with hypotension. As a result, low blood pressures will be significantly overestimated; in some cases the relative error can be greater than 10%. It is therefore important to correct the cuff pulse pressure measurement for the cuff compliance at the specific cuff pressure.

FIGS. 3a, 3b and 3c present a model of the cuff measurement principle. FIG. 3a shows an electrical model of the cuff around the arm (it is well known in the art that the electrical and mechanical domain are equivalent, and in practice it is often easier to analyse mechanical systems in the electrical domain). The arm-plus-artery system (C_arm_artery) and the cuff (C_Cuff) are both modelled as variable compliances (represented by non-linear capacitances in the electrical model). FIG. 3b shows a typical volume-transmural pressure curve of the arm-plus-artery and FIG. 3c shows a typical volume-pressure relation of the cuff. It can clearly be seen from FIGS. 3b and 3c that the cuff compliance is much bigger than the arm-plus-artery compliance (i.e. the cuff experiences a much greater volume change for a similar pressure change).

The volume of the arm-plus-artery changes in dependence on the transmural pressure over the arm (where the transmural pressure is given by P_{BloodPressure}−Pₛₖᵢₙ, the internal pressure blood pressure as modelled by the voltage source 30 minus the external skin pressure). FIG. 3b shows that, in the illustrated example, a typical oscillation amplitude is about 0.1 ml (at a blood pressure of 120/80, when the external skin pressure is zero). The measurement cuff is modelled as another (variable) compliance in series with the arm-plus-artery. The cuff compliance can be modelled as a parallel combination of three compliances: (1) the compliance due to the air in the cuff (Cₐᵢᵣ), (2) the compliance due to the cuff elasticity (C_{cel}), and (3) the compliance due to the elasticity of the arm tissue (Cₐᵣₘ).

During a cuff measurement, the pump 21 (represented by current source 31 in FIG. 3a) causes air to be pumped into the cuff. As a result the volume of the cuff is increased and the volume of the arm-plus-artery is decreased. The effect on the volume of the cuff is significantly greater than the effect on the volume of the arm-plus-artery, because of the significantly greater compliance of the cuff. During cuff inflation the pressure in the cuff increases, while the transmural pressure over the arm-plus-artery decreases. The change in transmural pressure results in a change of volume in the arm-plus-artery. In the example illustrated by FIG. 3b (in which the blood pressure is 120/80), when 50 mmHg cuff pressure is applied the volume change dV of the arm-plus-artery is 1.05−0.75=0.3 ml (i.e. the volume change corresponding to a pressure change dP of 120/80−50=70/30 mmHg). However, in oscillometric blood pressure measurement it is the blood volume oscillation amplitude changes which are, in principle, the target measurement. Although it is actually the cuff pressure oscillations that are measured, it is assumed that these are a true representation of the arm-plus-artery volume changes (i.e. it is assumed that the transfer function of volume change to pressure change is constant over the clinically relevant range).

The small (˜0.1 ml to 1 ml) volume changes of the arm-plus-artery are transmitted from the arm into the cuff, where these volume changes result in a pressure change in the cuff. These pressure changes are small, because the compliance of the cuff is much bigger than that of the arm-plus-artery (as can be seen from FIG. 3c, a 0.1 ml-1 ml volume change translates to a very small pressure change).

Clearly, a distortion of the shape of the envelope of the high-pass filtered cuff pressure oscillation amplitude will cause systematic errors in estimated blood pressures, because the pressures corresponding with the required amplitude points for systole and diastole will be altered due to the distortion. The cuff pressure and volume changes are related by:

$$\frac{{dV}_{a}}{dt} = {C}_{QS} ( {P}_{C} ) \cdot \frac{{dP}_{C}}{dt} \qquad ( 2 )$$

where Vₐ is the change in arm volume due to artery volume pulsations (in units of ml), C_{QS} is the pressure-dependent QS cuff compliance (in units of ml/mmHg), and P_{C} is the measured cuff pressure. Vₐ is time dependent due to the varying artery-cuff transmural pressure. When the cuff compliance is constant the volume and pressure changes are proportional to each other, and hence the ratio does not depend on cuff pressure. However, when cuff compliance is pressure dependent the differential equation needs to be solved.

Compliance data for a given cuff which has been obtained under controlled conditions (such as the data shown in FIG. 4) cannot be used in a lookup table or in a feed forward mode to correct oscillatory NIBP measurements because the cuff compliance is affected by the tightness of the wrapping of the cuff, the arm diameter, and the mechanical properties of the arm (e.g. the amount of soft tissue, the soft tissue pressure dependent compliance, changes to soft tissue properties due to hysteresis and/or previous measurements). Cuff compliance must therefore be measured during the actual NIBP measurement.

In electronic engineering the small-signal method is commonly used to approximate the behaviour of non-linear devices with linear equations. In this method a DC bias is applied to a device, and a small AC signal is superimposed on the DC voltage. A voltage dependent capacitance can thereby be measured. This method has been applied to measuring cuff compliance; however it has the drawback of requiring a special high frequency pump and a different valve arrangement. Furthermore, this method is susceptible to error because of the RC filter characteristics of the cuff-tube combination and because air and cuff volume changes are not the same due to the compressibility of air. A mass flow sensor must therefore be used.

Other methods to determine the pressure dependent cuff compliance are described in U.S. Pat. No. 5,103,833, U.S. Pat. No. 6,039,359 and U.S. Pat. No. 6,309,359. However, these methods all suffer from significant drawbacks. In particular, they are not applicable for all types of NIBP devices; they require major hardware changes (e.g. special pumps, sensors, flow meters); and in some cases the measurement errors are large. Furthermore, these methods were employed to determine properties of the brachial arteries and not to measure blood pressure. U.S. Pat. No. 8,308,648 describes a method in which transfer characteristics (due to pressure dependent cuff compliance) are used to correct the pressure envelopes used in oscillometric NIBP. However, this method also requires specialized hardware (a rigid container, two pressure bladders, a bladder with fixed volume), and is therefore not suitable for use with conventional NIBP devices and is not compatible with conventional patient monitors.

Besides cuff compliance, the flow resistance of the tubing 27 can also give rise to errors. This can be due to Ohmic pressure drops during ramps, or due to RC filtering effects on the rapidly changing flows and pressures.

Since the absolute value of blood pressure is of clinical importance to determine if a subject has a hyper- or hypotension, a convenient way of acquiring NIBP measurements at a reduced error would be a valuable tool. There is therefore a need for an improved method and apparatus that can acquire oscillatory NIBP measurements having significantly greater accuracy than the conventional oscillometric method, whilst also being compatible with conventional NIBP devices and patient monitors.

### SUMMARY OF THE INVENTION

It is the aim of the present invention to reduce or eliminate errors resulting from variations in the characteristics of the cuff, the cuff placement and in the size and mechanical properties of the arm between different subjects from oscillatory NIBP measurements. Certain embodiments of the invention also seek to reduce or eliminate errors resulting from tube flow resistance and pressure drop over the tube. It is an additional aim of the invention to provide an improved oscillatory NIBP apparatus and method which is backwards compatible with existing devices used in the measurement of NIBP.

Therefore, according to a first aspect of the invention, there is provided a method for use in cuff-based oscillatory non-invasive blood pressure, NIBP, measurement, the method comprising:
progressively altering the volume of air in a cuff of a NIBP measurement apparatus during a measurement period;
   obtaining a plurality of measurements of the flow rate of the air into/out of the cuff during the measurement period;
obtaining a plurality of measurements of the air pressure in the cuff during the measurement period; and
determining a relationship between quasi-static cuff compliance and cuff pressure by calculating the quasi-static cuff compliance at a plurality of instances during the measurement period, based on the flow rate measurements and the air pressure measurements obtained during the measurement period.

Embodiments of the invention permit reduction or elimination of errors in the blood pressure estimation which are due to distortion of the pulse pressure envelope resulting from non-constant QS cuff compliance.

Advantageously, only minor changes to the hardware of a conventional NIBP device are required to implement embodiments of the invention. This means that embodiments of the invention enable pressure dependent cuff compliance to be determined during a normal NIBP measurement using a conventional single lumen cuff. Some embodiments also enable tube resistance to be determined during a normal NIBP measurement using a conventional single lumen cuff.

Some advantageous embodiments permit the measurement time to be shortened. For example, by combining measurements corrected for cuff compliance and tube flow resistance acquired during both ramp up and ramp down of cuff pressure, a faster ramp rate can be used and the overall measurement time can be reduced.

In some preferred embodiments of the invention the measurement period comprises an inflation period during which the volume of air in the cuff is progressively increased and a deflation period during which the volume of air in the cuff is progressively decreased. In some such embodiments, the rate at which the volume of air in the cuff is altered during the inflation period is different to the rate at which the volume of air in the cuff is altered during the deflation period. In some such embodiments the volume of air in the cuff is altered during the deflation period is non-constant. In some such embodiments the volume of air in the cuff is altered in a step-wise manner during the deflation period.

In some embodiments the method further comprises using the obtained flow rate measurements to determine the resistance of a tube passed through by air flowing into or out of the cuff. In some such embodiments progressively altering the volume of air in the cuff during the measurement period comprises controlling a flow of air into the cuff such that the pressure in the cuff increases at a predetermined rate during the inflation period and subsequently controlling a flow of air out of the cuff such that the pressure in the cuff decreases at a predetermined rate during the deflation period. In such embodiments determining the tube resistance comprises:
calculating the volume of the cuff at a plurality of instances during each of the inflation period and the deflation periods; and
calculating a difference between the cuff pressure at a given volume during the inflation period and the cuff pressure at the give volume during the deflation period.

In some embodiments the rate at which the volume of air in the cuff is altered during the measurement period is selected such that the measurement period includes at least a predefined minimum number of heartbeats of the subject. In some such embodiments the predefined minimum number of heartbeats is ten heartbeats. Advantageously, embodiments which define a minimum number of heartbeats ensure that an accurate blood pressure value can be obtained whilst minimizing the measurement time as far as possible.

In some embodiments the NIBP measurement apparatus is arranged to acquire a measurement of the blood pressure of a subject. In such embodiments the method further comprises calculating one or more of: a systolic blood pressure of the subject, a diastolic blood pressure of the subject and a mean blood pressure of the subject, based on the air pressure measurements obtained during the measurement period and on the determined relationship between quasi-static cuff compliance and cuff pressure. In some such embodiments the calculating is additionally based on the determined tube resistance.

In some embodiments the rate at which the pressure in the cuff is altered during the measurement period is greater than 10 mmHg/s. Advantageously, embodiments of the invention can compensate for the tube resistance errors incurred at higher ramp rates, enabling measurement time to be reduced without lowering measurement accuracy.

There is also provided, according to a second aspect of the invention, a control unit for a NIBP measurement apparatus having an inflatable cuff for wrapping around a body part of a subject. The control unit comprises:
at least one output for sending control signals to the NIBP measurement apparatus and to a flow meter;
at least one input for receiving measurements from the NIBP measurement apparatus and from the flow meter; and
a processing unit configured to:
control the NIBP measurement apparatus to progressively alter the volume of air in a cuff during a measurement period and to obtain a plurality of measurements of the air pressure in the cuff during the measurement period;
control the flow meter to obtain a plurality of measurements of the flow rate of the air into/out of the cuff during the measurement period;
receive the air pressure measurements obtained by the NIBP measurement apparatus and the flow rate measurements obtained by the flow meter; and
   determine a relationship between quasi-static cuff compliance and cuff pressure by calculating the cuff compliance at a plurality of instances during the measurement period, based on the received flow rate measurements and the received air pressure measurements.

In some embodiments the processing unit is further configured to control the NIBP measurement apparatus to progressively alter the volume of air in a cuff during a measurement period at a given rate. In some such embodiments the processing unit is configured to control the NIBP measurement apparatus to progressively alter the volume of air in a cuff during a measurement period at a first rate during a first part of the measurement period and at a second, different, rate during a second part of the measurement period.

There is also provided, according to a third aspect of the invention, a system for use in oscillatory non-invasive blood pressure, NIBP, measurement. The system comprises:
a NIBP measurement apparatus having an inflatable cuff for wrapping around a body part of a subject;
a flow meter configured to measure the flow rate of air into/out of the cuff; and
a control unit according to the second aspect of the invention.

In some embodiments the flow meter comprises at least one pressure sensor and the NIBP apparatus comprises at least one pressure sensor, and at least one pressure sensor comprised in the flow meter is also comprised in the NIBP measurement apparatus. In some such embodiments the flow meter comprises two pressure sensors and the NIBP measurement apparatus comprises two pressure sensors, and the two pressure sensors of the flow meter are the same as the two pressure sensors of the NIBP measurement apparatus. Such embodiments advantageously mean that a conventional NIBP device can be used to implement the invention, with very little modification.

There is also provided, according to a fourth aspect of the invention, a computer program product, comprising computer readable code embodied therein, the computer readable code being configured such that, on execution by a suitable computer or processor, the computer or processor operates as a control unit according to the second aspect of the invention.

### BRIEF DESCRIPTION OF THE DRAWINGS

For a better understanding of the invention, and to show more clearly how it may be carried into effect, reference will now be made, by way of example only, to the accompanying drawings, in which:

FIG. 1 is a graph of cuff pressure versus time measured using a conventional oscillometric method and apparatus;

FIG. 2 shows a graphical overview of the main elements in a conventional oscillatory NIBP measurement apparatus;

FIG. 3a is a circuit diagram relating to a conventional oscillatory NIBP measurement apparatus;

FIG. 3b is a graph showing the volume-transmural pressure relation of an exemplary arm-plus-artery system;

FIG. 3c is a graph showing the volume-cuff pressure relation of an exemplary cuff;

FIG. 4 is a graph of cuff compliance versus cuff pressure for an exemplary cuff;

FIG. 5 shows a graphical overview of the main elements of the NIBP apparatus according to an embodiment of the invention;

FIG. 6 shows a method for use in oscillatory NIBP measurement according to a first embodiment of the invention;

FIG. 7 is a graph of cuff volume versus cuff pressure for an exemplary cuff;

FIG. 8 is a graph of cuff compliance versus cuff pressure for the exemplary cuff of FIG. 7 obtained using two different methods;

FIG. 9a is a graph showing uncorrected and corrected normalized volume envelopes for a first subject;

FIG. 9b is a graph showing uncorrected and corrected normalized volume envelopes for a second subject;

FIG. 10 shows a method for use in oscillatory NIBP measurement according to a second embodiment of the invention; and

FIG. 11 shows a hysteresis loop used to extract tube resistance in certain specific embodiments of the invention.

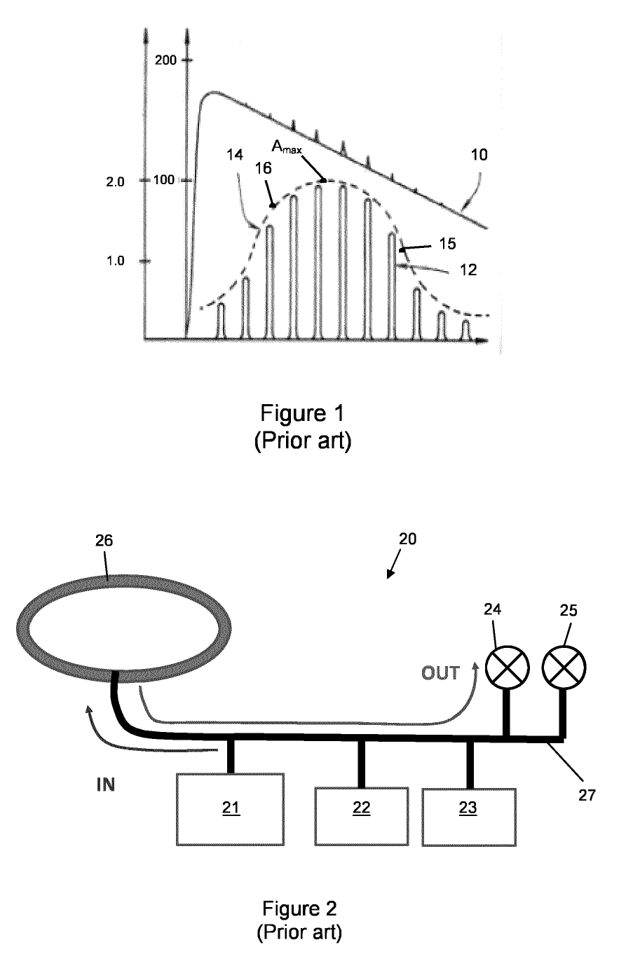

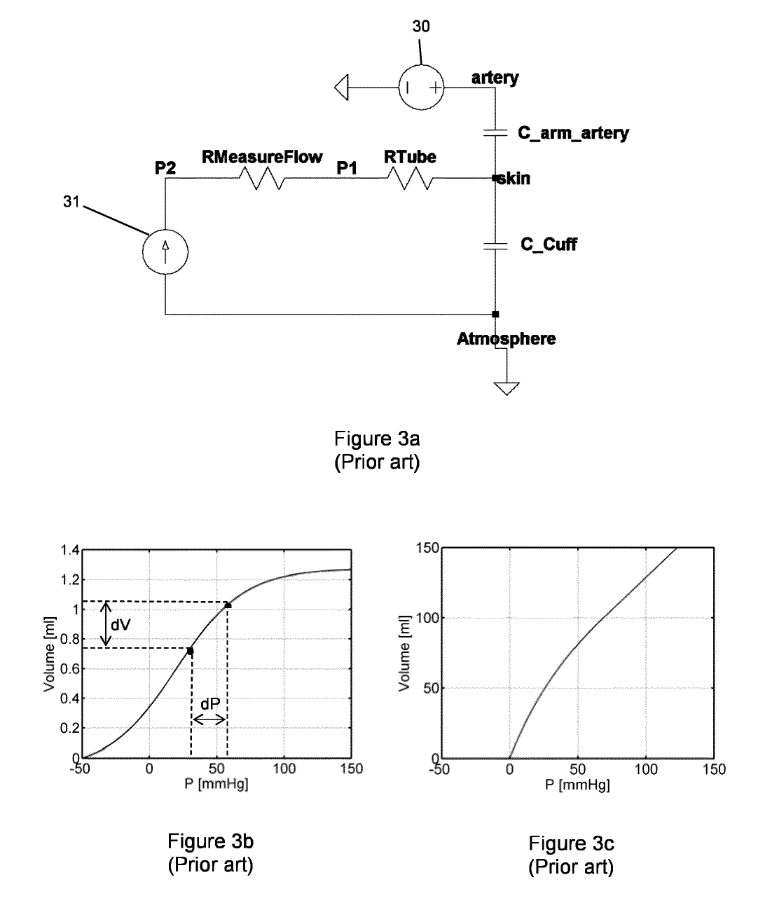

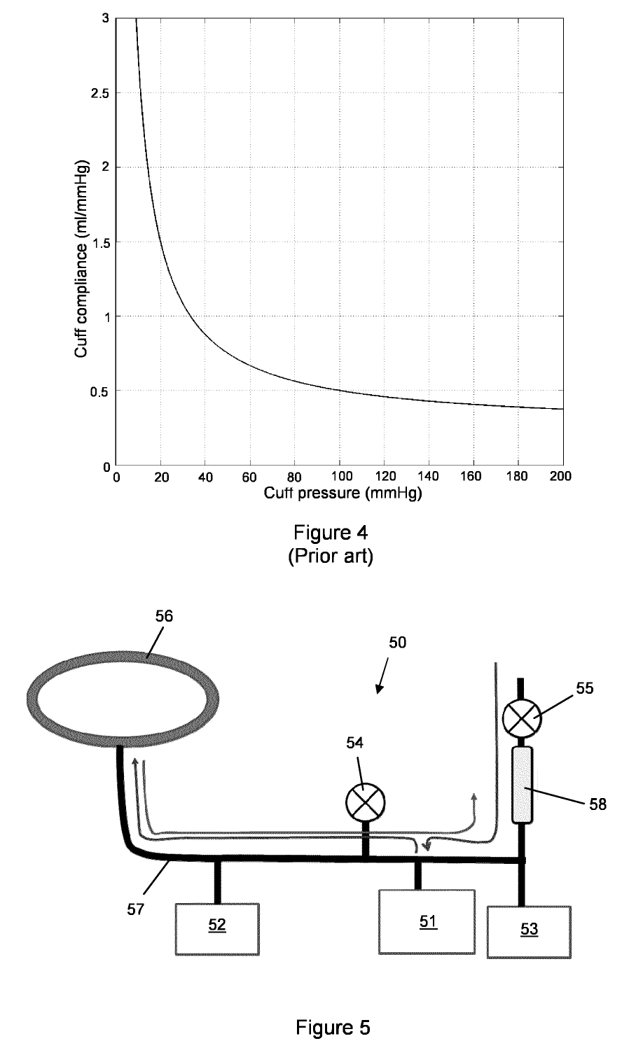

### DETAILED DESCRIPTION OF THE PREFERRED EMBODIMENTS

Embodiments of the invention use a quasi-static method to measure cuff compliance during an NIBP measurement. A cuff compliance curve specific to that measurement is thereby generated, and is used to correct the pressure envelope. The large relative error in blood pressure measurement due to non-constant cuff compliance and changing cuff compliance between measurements is thereby reduced or eliminated.

FIG. 5 shows an apparatus 50 for use in oscillatory NIBP measurement that is suitable for implementing the method according to the invention. As can be seen from a comparison with FIG. 2, the apparatus 50 comprises the same components as a conventional oscillatory NIBP measurement device, namely a pump 51, first and second pressure sensors 52, 53, and first and second valves 54, 55, connected to a cuff 56 by tubing 57. However, the apparatus 50 is configured such that the air volume flow between the pump 51 and the cuff 56 can be measured in both directions. The layout of the tubing 57 has been modified from the conventional layout shown in FIG. 2, such that the first pressure sensor 52 is between the pump 51 and the cuff 56 and the first valve 54 is between the first pressure sensor 52 and the pump 51. Furthermore, a flow limiting element 58 (e.g. a Venturi element, a flow resistor, an orifice, etc.) has been inserted between the second pressure sensor 53 and the second valve 55. With this arrangement the air volume flow through the tubing 57 can be determined by using the second pressure sensor 53 and the resistance value of the flow limiting element 58. Thus in the apparatus 50 the pressure sensors 52, 53 have a dual function during measurement—they are used both for sensing cuff pressure and for measuring air volume flow. It will be appreciated that this arrangement allows a flow sensor to be realized with minimum changes to the hardware of a conventional NIBP device. However, alternative embodiments are possible in which the first and second pressure sensors 52, 53 are replaced by a differential pressure sensor.

Alternative embodiments are also possible in which a conventional oscillatory NIBP measurement arrangement as shown in FIG. 2 is used, together with a pump having known pumping characteristics (i.e. known flow versus output pressure). In such embodiments, the method of the claimed invention can only be performed during inflation of the cuff (by contrast, the apparatus shown in FIG. 5 enables measurement of the cuff compliance during both inflation and deflation of the cuff).

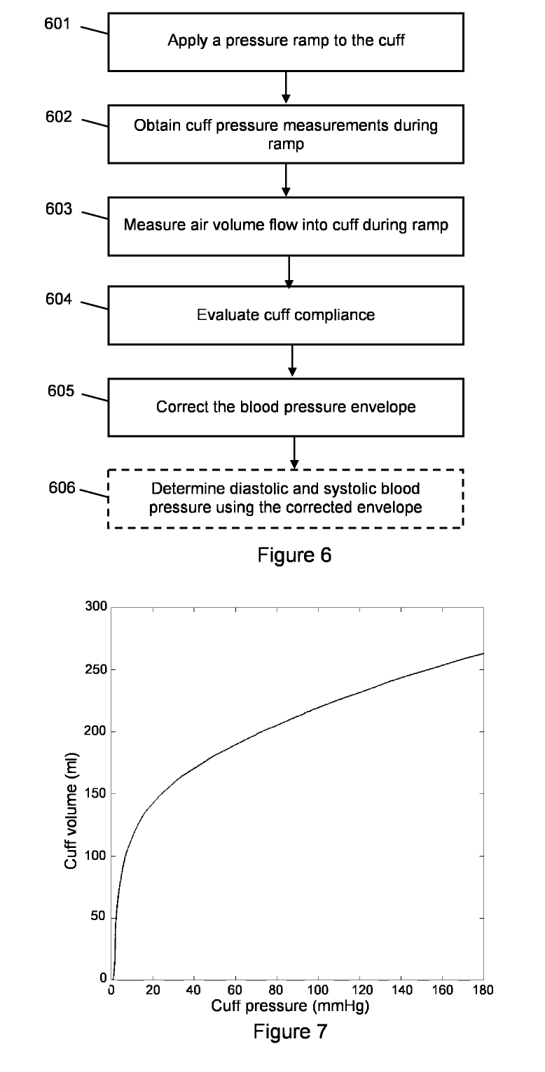

FIG. 6 shows a method for use in oscillatory NIBP measurement according to a first embodiment of the invention. In step 601 a pressure ramp is applied to the cuff, to reach a cuff pressure above systolic blood pressure. In preferred embodiments the pressure ramp is sufficiently slow (˜5 mmHg/s) for the method to be quasi-static. In some embodiments the ramp is upwards (i.e. the cuff pressure increases over the course of the ramp). In alternative embodiments the ramp is downwards (i.e. the cuff pressure decreases, starting from above systolic, over the course of the ramp). In some embodiments two pressure ramps are applied (e.g. an upwards ramp corresponding to inflation of the cuff by the pump followed by a downwards ramp corresponding to deflation of the cuff through one or more of the valves).

In step 602 cuff pressure measurements are obtained periodically during the pressure ramp, in the conventional manner. In some embodiments in which two pressure ramps are applied, cuff pressure measurements are obtained periodically during both pressure ramps.

In step 603 the air volume flow into the cuff during the pressure ramp is measured. This airflow is measured by measuring the pressure drop (with the second pressure sensor 53) over the flow limiting element 58:

\dot{V}ₛ=(Pₛ−P_{ambient})/R (3)

where \dot{V}ₛ is the air volume flow rate under standard conditions (i.e. atmospheric pressure and ambient temperature), Pₛ is standard (i.e. atmospheric) pressure, P_{ambient} is ambient pressure, and R is the resistance of the flow limiting element. The volumetric air flow at atmospheric pressure and ambient temperature is converted to volume flow at cuff pressure using:

$${\dot{V}}_{C} = {\dot{V}}_{s} \cdot {[ \frac{{P}_{s}}{{P}_{C}} ]}^{\frac{1}{γ}} \qquad ( 4 )$$

where \dot{V}_{C} is the air volume flow rate into the cuff, P_{C} is the cuff pressure, and γ is a constant which takes a value of 1 for an isothermal process and a value of 1.4 for an adiabatic process. In embodiments in which the method is quasi-static (i.e. for typical use in methods according to the invention), γ is approximately equal to 1. \dot{V} and \dot{P} are alternative notations for the time derivative of the air volume dV/dt and the time derivative of the pressure dP/dt respectively. The flow pressure sensor should measure absolute pressure, because the pressures in equation 4 are absolute pressures.

In step 604 the cuff compliance is evaluated using the following procedure. The complete pressure change over time dP/dt during the ramp is known (for example, because the pressure is measured and converted into the digital domain by an analogue-to-digital converter so that the time series of pressure-time is automatically available, and numerical differentiation methods can then be applied to obtain dP/dt), and the air volume flow into the cuff \dot{V}_{C} is known from step 603. It is assumed that the cuff bladder volume can be neglected at the start of the pressure ramp up (alternatively, this volume is known). The cuff volume at time t is obtained by integration of the air volume flow \dot{V}_{C} (this includes the air volume in the tube). In some embodiments the measured cuff pressure and air flow data is low pass filtered (for example using f_{c}=0.5 Hz). In such embodiments the cuff volume is calculated using the low pass filtered data. If artefacts (due to, e.g., outliers, missing beats, arrhythmias, etc.) are present in the measured data, appropriate corrections can be applied.

When the pressure ramp is slow (and hence the volume flow rate is relatively low), the effect of tube resistance on flow and pressure can be neglected and the cuff pressure is therefore assumed to be equal to the pressure measured by the apparatus 50. The quasi-static cuff compliance is then calculated from:

C_{QS}=\dot{P}_{C}/\dot{V}_{C} (5)

where C_{QS} is the QS cuff compliance and \dot{P}_{C} is the time derivative of cuff pressure.

Alternatively, when V_{C} and P_{C} are known (for example from pressure measurements and an integration of air flow measurements, as described above) the QS cuff compliance at pressure P_{C} can be estimated from the known cuff volume-pressure relation using:

$${C}_{QS} ( {P}_{C} ) = {( \frac{\partial {V}_{C}}{\partial {P}_{C}} )}_{{P}_{C}} + {C}_{C} \qquad ( 6 )$$

In preferred embodiments the output of step 604 is a data set relating cuff compliance to pressure across the whole pressure range of the ramp up. This data set can then be used to determine a relationship between quasi-static cuff compliance and cuff pressure, using known mathematical techniques. In preferred embodiments the determined relationship has the form:

$${\dot{V}}_{C} = {C}_{QS} \cdot \frac{{dP}_{C}}{dt} \qquad ( 7 )$$

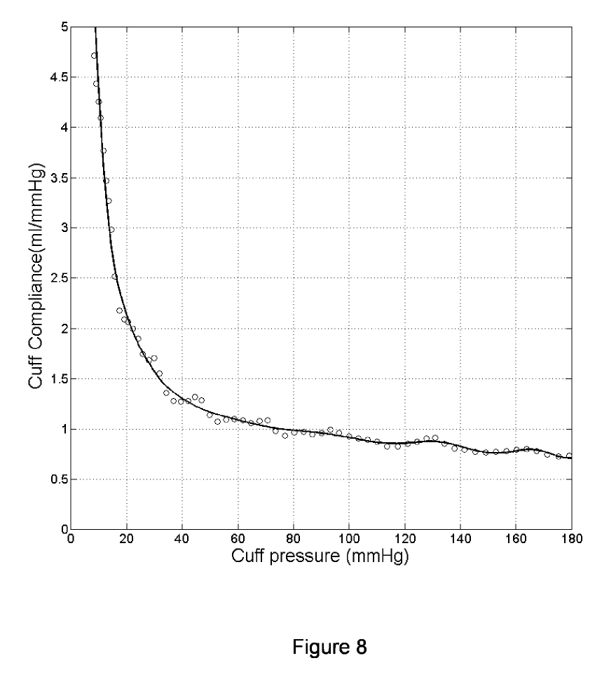

To validate the accuracy of the method, the cuff compliance can be calculated using equation 5 and using equation 6, and the results of the two calculations compared for consistency. Experiments performed by the current inventors have demonstrated that the quasi-static method provides accurate measurements for the cuff compliance which are free from high frequency artefacts and that can be done during the normal oscillometric NIBP blood pressure measurement. FIG. 7 shows the measured static volume-pressure curve obtained in these experiments for a particular adult cuff on an arm. FIG. 8 shows the cuff compliance of the same cuff obtained using the quasi-static method (solid line) and using the static volume-pressure curve of FIG. 7 together with equation 6 (dots). It can be seen that the measured cuff compliance obtained using the quasi-static method agrees well with that obtained using the measured static volume-pressure curve.

In step 605 the QS cuff compliance-cuff pressure relationship determined in step 604 is used to correct the blood pressure envelope, in the following manner. First, a pressure envelope is derived from the cuff pressure measurements obtained in step 603 using conventional techniques. In some embodiments the cuff-pressure is low-pass filtered to remove high-frequency artefacts (e.g. using a bandwidth of ˜25 Hz) and then high-pass filtered to remove the DC and slow ramp components (e.g. using a cut off frequency of ˜0.25 Hz) and the pressure envelope is derived from this filtered signal. In some embodiments, artefacts (due to, for instance, arrhythmias) are removed at this stage.

In some embodiments correction of the envelope for cuff compliance is done by numerical integration of equation 7. Alternatively, when cuff compliance variation is small in the specific cuff pressure range, the correction can be done using ΔV(P)˜ΔP_{osc}*C_{QS}(P_{C}). A corrected envelope having dimensions of volume is thereby generated. This curve can be normalized to dimensionless units (as is done for the pressure curve) by dividing the volume oscillations by the maximum volume oscillation. In some embodiments (e.g. embodiments in which a model of the cuff compliance and arterial volume as function of transmural pressure is known) the envelope correction can be enhanced using curve fitting methods. It will be appreciated that the person skilled in the art will be aware of various mathematical techniques which could alternatively be employed in the correction of the envelope.

It will also be appreciated that the QS cuff compliance-cuff pressure relationship determined in step 604 can beneficially be applied in ways which do not involve correcting a blood pressure envelope. For example, it could be used to compare the compliance behaviour of different cuff designs or brands, and/or to train medical personnel to wrap cuffs in a manner to as to minimize compliance variation. Various other applications will be readily apparent to the skilled person.

The corrected envelope can be used to determine the diastolic and systolic blood pressure in a conventional manner (shown as an optional step 606 in FIG. 6). This procedure is not affected by the units of the envelope because it uses dimensionless ratios.

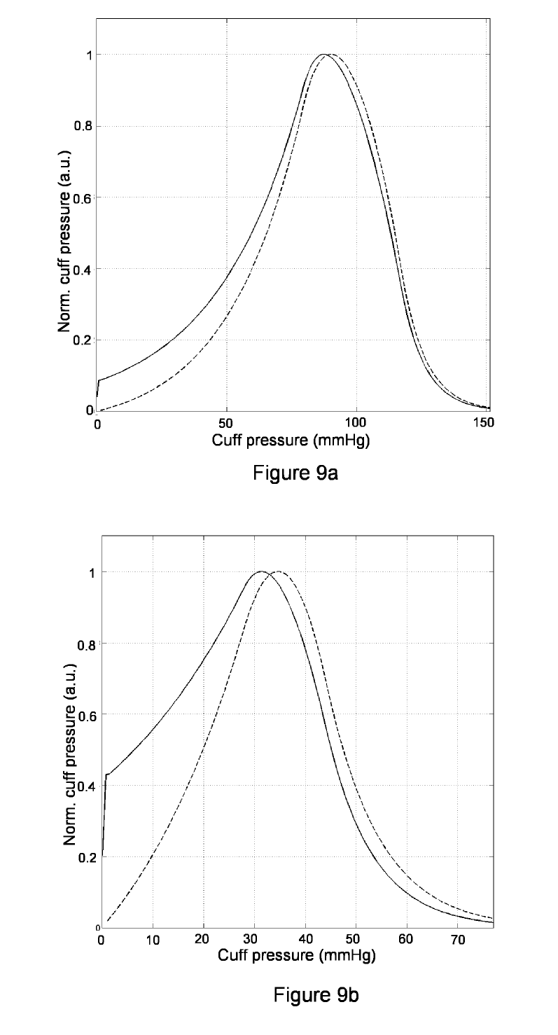

FIGS. 9a and 9b show the results of a simulation which illustrates the impact of the envelope correction on blood pressure estimation for a subject with normal blood pressure (˜80/120 mmHg) and a severely hypotensive subject (blood pressure˜30/50) respectively. The simulation uses a brachial artery volume-pressure relation from Jeon et. al., World Acad. Sci. Eng. Technol. 2007, 30: 366-371. In each of FIGS. 9a and 9b the dashed curve is the uncorrected pressure envelope and the solid curve is the corrected pressure envelope. It can be seen that the corrections are negligible for the normotensive patient (˜2 mmHg), but for the hypotensive case the corrections are ˜6 mmHg, which is large compared to the measured value. It can also be seen that, besides the changes of systole and diastole, the maximum point of the curve (which in many cases is used as mean blood pressure) is also shifted. The deviations from actual values of the calculated values for the systolic, mean and diastolic blood pressure values based on the uncorrected envelope are clinically relevant (˜20%). When the corrected envelope is used, the deviations are significantly smaller.

Thus, the method in FIG. 6 enables errors in oscillatory NIBP measurements resulting from variable cuff compliance to be reduced or even entirely eliminated. This is achieved by measuring the cuff compliance for each individual NIBP measurement performed, to obtain cuff compliance data specific to that particular measurement. This data is then used to generate a blood pressure envelope which is corrected for the effects of varying cuff compliance. Blood pressure values estimated using the corrected envelope can therefore be significantly more accurate than blood pressure values estimated using conventional techniques. Furthermore, the method can be implemented by a conventional NIBP device following only minimal changes to its hardware, and does not increase the time or complexity of performing a blood pressure measurement.

At relatively high volume flow rates, or if relatively long and/or narrow tubing is used, the resistance of the tubing to air flow (hereafter referred to as the tube resistance) results in a pressure drop over the tube that can no longer be neglected. This causes a significant additional error (in the range 1 to 10 mmHg), which can be corrected when the tube resistance is known. Like cuff compliance, tube resistance should be measured during a NIBP measurement because tube resistance is affected by the temperature, and by the exact path of the tubing (i.e. by any bends or curves in the tubes).

Tube resistance can be estimated from air volume flow rate, therefore embodiments of the invention also enable oscillatory NIBP measurements to be corrected for tube resistance. This means that embodiments of the invention can use a higher ramp rate without reducing the accuracy of the resulting blood pressure measurements, consequently allowing a blood pressure measurement to be acquired in a shorter time. It will be appreciated that the measurement time cannot be made arbitrarily short, as a minimum number of heart beats (˜10) must be recorded to enable calculation of the blood pressure envelope. However, if air pressure and flow measurements are acquired during ramp up and ramp down, the ramp rate can be increased as compared to the conventional method (e.g. since 5 heat beats can be recorded during ramp up and 5 heart beats can be recorded during ramp down). As a result the total measurement time can be reduced.

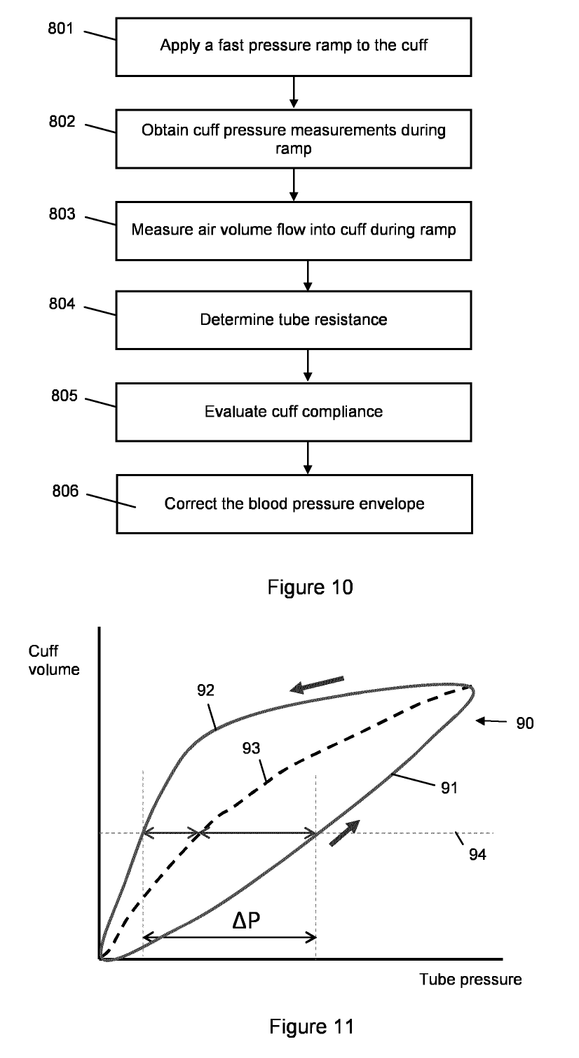

Accordingly, FIG. 10 shows a method for use in oscillatory NIBP measurement according to a second embodiment of the invention. This method assumes that the flow resistance of the tube is constant (i.e. the tube lumen diameter is constant) during the NIBP measurement. In step 801 a fast (˜10-20 mmHg/s) pressure ramp is applied to the cuff. In step 802 cuff pressure measurements are obtained periodically during the pressure ramp, in the conventional manner. In step 803 the air volume flow into the cuff during the pressure ramp is measured, as described in relation to step 603 of FIG. 6.

In step 804 the tube resistance is determined using one of several possible methods. Three such methods are described:

Flow Transient after Start of a Deflation Period.
In cases where the inflation of the cuff is pressure controlled, the pressure in the tubing 57 is measured (and controlled) and the pressure in the cuff P_{C} is given by:

P_{C}=P_{Tube}−\dot{V}*R_{Tube} (8)

where P_{Tube} is the pressure in the tubing 57 and R_{Tube} is the resistance of the tubing 57. At the end of the ramping period, when the flow is zero, P_{Tube} and P_{C} are equal. Then the cuff is deflated by opening one of the first and second valves 54, 55. The pressure drop during deflation from the cuff to the exit of the open valve is given by:

ΔP=P_{C}−Pₐₜₘ. (9)

The sum R of the tube resistance and the (known) internal resistance of the blood pressure device (e.g. parasitic resistance due to valves etc.) can now be calculated using:

$$R = \frac{Δ P}{Δ \dot{V}} \qquad ( 10 )$$

where R=R_{Tube}+Rᵢₙₜ. At high cuff pressure the flow rate is very high (˜1 l/s), and this can cause measurement artefacts (e.g. due to turbulence, non-linearity, etc.). For this high flow case it is preferable to analyse the pressure-time data in terms of a low-pass RC network. RC time can be determined, since cuff compliance is almost constant at high pressure and is known from the ramp up phase. R can be determined in this pressure range. In some embodiments discrete deflation steps at lower cuff pressure are used. In such embodiments, the pressures (and hence the peak flows) are lower and so the measurements can be more accurate. Subtracting the known internal resistance Rᵢₙₜ from R gives R_{Tube}. In some alternative embodiments, the pressure measurements acquired during inflation of the cuff are used to calculate R.
Method with Flow Control or Known Initial Flow
In cases where the air flow is controlled (e.g. because the pump 51 is a fixed flow pump, or alternatively is servo controlled) R_{Tube} can be estimated at the end of the ramping period. When the air flow stops, the pressure measured in the tubing 57 will drop because the pressure drop over the tube vanishes. From the observed pressure drop and the known air flow at the end of the ramp up the tube resistance can be estimated using equation 9. A drawback of this method is a second pressure drop due to mechanical hysteresis of the cuff. Consequently, only the fast transient pressure drop should be considered. In some alternative embodiments this method is applied in the initial phase of the cuff inflation. In such embodiments operating the pump intermittently and measuring the resulting changes in pressure allows the tube resistance to be measured (using Ohms law), since the flow is known.

### Method Based on the Cuff Volume—Pressure Hysteresis Loop

In methods according to the invention, pressure in the tubing 57 (P_{Tube}) and air volume flow into the cuff 56 (\dot{V}) are both measured with high accuracy during inflation and deflation of the cuff 56. Cuff volume V_{C} is then obtained by integration, as explained above in relation to step 604 of FIG. 6. When the calculated V_{C} is plotted against P_{Tube} a hysteresis loop is observed. An example of such a loop 90 is shown in FIG. 11 (in which V_{C} is on the y-axis and P_{Tube} is on the x-axis). In FIG. 9 the lower part 91 of the loop represents inflation and the upper part 92 of the loop represents deflation. The dashed line 93 is the static cuff volume-cuff pressure relation. The hysteresis loop is partly caused by the flow resistance of the tubing 57 (other contributions come from cuff material hysteresis and increases in arm volume due to blockage of venous flow). To reduce the effects of mechanical hysteresis and change in arm volume, the deflation should be fast.

When it is assumed that tube resistance depends only on the air volume in the cuff-tube system the tube resistance R_{Tube} at a given cuff volume is the same for both inflation and deflation. The current method for determining tube resistance exploits this to thereby enable the determination of both the pressure dependence of R and the cuff compliance in a single step, using the following procedure. (This procedure assumes that the tube and cuff are purely elastic and that arm volume changes due to blood pooling in the arm are small—ramp down should therefore be fast, as mentioned above). In this example cuff pressure is servo controlled during continuous ramp up and ramp down; however, this method also works with different implementations of pressure and volume control.

The cuff is inflated and deflated, at predetermined ramp rates using a servo controlled system (tube pressure control). In preferred embodiments the ramp down rate is significantly faster than the ramp up rate. The air flow and air pressure signals are low pass filtered and artefacts are removed. The cuff volume is obtained by integrating the processed air flow over time, and a P-V hysteresis loop is measured as described above. The tube resistance for a selected cuff volume (e.g. the volume represented by the horizontal dashed line 84 in FIG. 8) can then be determined from:

$$R = \frac{( flow 1 + flow 2 )}{Δ P} \qquad ( 11 )$$

where flow1 and flow2 are the absolute values of the air flow at inflation and deflation for the selected cuff volume and AP is the difference in tube pressure between the forward and backward flows. Using this method it is also possible to determine the volume dependence of the tube resistance (by its dependence on pressure) and the corrected cuff volume-pressure relation using the unknown cuff volume and cuff pressures.

When all of the tube resistance, the cuff pressure, and the cuff volume are known, the cuff compliance can be estimated from the static V-P curve. In preferred embodiments, the estimation is done using measurements from the extremes of the hysteresis loop (i.e. the highest and lowest pressures) with shortest possible delay time to reduce the impact of arm volume changes and cuff hysteresis. Deflation should be fast for the same reasons. Hence the hysteresis loop method enables both cuff compliance and tube resistance to be obtained in a single measurement.

In step 805 the cuff compliance is evaluated. Once R is known (e.g. from one of the above methods) it is possible to calculate the actual cuff pressure P_{C}(t) from:

P_{C}=P_{Tube}−\dot{V}·R (12)

Since the air flow and the pressure in the tubing are known, the cuff compliance C_{C} can be determined for all cuff pressures P_{C} as described above in relation to step 604 of FIG. 6. Hence the oscillatory NIBP measurement can be corrected for both tube resistance and cuff compliance for an arbitrary single lumen cuff, at fast inflation and deflation rates.

In step 806 the cuff compliance data calculated in step 805 and the tube resistance calculated in step 804 are used to correct the blood pressure envelope, using the procedure described above in relation to step 605 of FIG. 6. The corrected envelope can then be used to determine the diastolic and systolic blood pressure in a conventional manner.

It is clear from the above that NIBP measurement can be done both fast and accurately using methods and apparatus according to the invention in which both tube resistance errors and cuff compliance transfer characteristics are accounted for. Being able to determine the tube resistance means that ramp rates can be significantly increased (up to a level where only 10 beats are observed per NIBP measurement). Furthermore, in some embodiments cuff pressure data is collected during both inflation and deflation, further decreasing the total time required for the measurement. This is advantageous as frequent NIBP measurements can be painful for the subject and can even cause them harm. In preferred embodiments measurement speed is determined by the number of beats (or cuff pressure pulses) required for a reliable blood pressure measurement. It will be appreciated that embodiments which enable faster and less obtrusive NIBP measurements are particularly suitable for applications where frequent blood pressure measurements are required (e.g. in hospitals, for ambulatory NIBP, etc.).

While the invention has been illustrated and described in detail in the drawings and foregoing description, such illustration and description are to be considered illustrative or exemplary and not restrictive; the invention is not limited to the disclosed embodiments.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A method for use in cuff-based oscillatory non-invasive blood pressure, NIBP, measurement, the method comprising:
progressively altering the volume of air in a cuff of a NIBP measurement apparatus during a measurement period;
obtaining a plurality of measurements of the flow rate of the air into/out of the cuff during the measurement period;
obtaining a plurality of measurements of the volume flow rate of the air into/out of the cuff during the measurement period;
obtaining a plurality of measurements of the air pressure in the cuff during the measurement period;
converting the measurements of the volume flow rate of the air into/out of the cuff into volume flow rate at cuff pressure, \dot{V}_{C}, using the measurements of the air pressure in the cuff;
determining the time derivative, \dot{P}_{C}, of the measurements of the air pressure in the cuff; and
determining a relationship between quasi-static cuff compliance and cuff pressure by calculating the quasi-static cuff compliance at a plurality of instances during the measurement period, based on the volume flow rate measurements and the air pressure measurements obtained during the measurement period, wherein the quasi-static cuff compliance is calculated as the ratio of \dot{V}_{C} to \dot{P}_{C}.

2. The method of claim 1, wherein the measurement period comprises an inflation period during which the volume of air in the cuff is progressively increased and a deflation period during which the volume of air in the cuff is progressively decreased.

3. The method of claim 2, wherein the rate at which the volume of air in the cuff is altered during the inflation period is different to the rate at which the volume of air in the cuff is altered during the deflation period.

4. The method of claim 2, wherein the rate at which the volume of air in the cuff is altered during the deflation period is non-constant.

5. The method of claim 1, wherein the method further comprises using the obtained flow rate measurements to determine the resistance of a tube passed through by air flowing into or out of the cuff.

6. The method of claim 5, wherein:
progressively altering the volume of air in the cuff during the measurement period comprises controlling a flow of air into the cuff such that the pressure in the cuff increases at a predetermined rate during the inflation period and subsequently controlling a flow of air out of the cuff such that the pressure in the cuff decreases at a predetermined rate during the deflation period; and
determining the tube resistance comprises:
calculating the volume of the cuff at a plurality of instances during each of the inflation period and the deflation periods; and
calculating a difference between the cuff pressure at a given volume during the inflation period and the cuff pressure at the given volume during the deflation period.

7. The method of claim 1, wherein the rate at which the volume of air in the cuff is altered during the measurement period is selected such that the measurement period includes at least a predefined minimum number of heartbeats of the subject.

8. The method of claim 1, wherein the NIBP measurement apparatus is arranged to acquire a measurement of the blood pressure of a subject, the method further comprising:
calculating one or more of: a systolic blood pressure of the subject, a diastolic blood pressure of the subject and a mean blood pressure of the subject, based on the air pressure measurements obtained during the measurement period and on the determined relationship between quasi-static cuff compliance and cuff pressure.

9. The method of claim 8, wherein the calculating is additionally based on the determined tube resistance.

10. The method of claim 1, wherein the rate at which the pressure in the cuff is altered during the measurement period is greater than 10 mmHg/s.

11. A control unit for a NIBP measurement apparatus having an inflatable cuff for wrapping around a body part of a subject, the control unit comprising:
at least one output for sending control signals to the NIBP measurement apparatus and to a flow meter;
at least one input for receiving measurements from the NIBP measurement apparatus and from the flow meter; and
a processing unit configured to:
control the flow meter to obtain a plurality of measurements of the volume flow rate of the air into/out of the cuff during the measurement period;
receive the air pressure measurements obtained by the NIBP measurement apparatus and the flow rate measurements obtained by the flow meter;
convert the measurements of the volume flow rate of the air into/out of the cuff into volume flow rate at cuff pressure, \dot{V}_{C}, using the measurements of the air pressure in the cuff;
determine the time derivative, \dot{P}_{C}, of the measurements of the air pressure in the cuff; and
determine a relationship between quasi-static cuff compliance and cuff pressure by calculating the cuff compliance at a plurality of instances during the measurement period, based on the volume flow rate measurements and the received air pressure measurements, wherein the quasi-static cuff compliance is calculated as the ratio of \dot{V}_{C} to \dot{P}_{C}.

12. A system for use in oscillatory non-invasive blood pressure, NIBP, measurement, comprising:
a NIBP measurement apparatus having an inflatable cuff for wrapping around a body part of a subject;
a flow meter configured to measure the flow rate of air into/out of the cuff; and
a control unit according to claim 11.

13. The system of claim 12, wherein the flow meter comprises at least one pressure sensor and the NIBP apparatus comprises at least one pressure sensor, and wherein at least one pressure sensor comprised in the flow meter is also comprised in the NIBP measurement apparatus.

14. The system of claim 13, wherein the flow meter comprises two pressure sensors and the NIBP measurement apparatus comprises two pressure sensors, and wherein the two pressure sensors of the flow meter are the same as the two pressure sensors of the NIBP measurement apparatus.

15. A computer program product, comprising computer readable code embodied therein, the computer readable code being configured such that, on execution by a suitable computer or processor, the computer or processor operates as a control unit according to claim 11.
